(19) United States
(12) Patent Application Publication
(10) Pub. No.: US 2014/0178920 A1
(43) Pub. Date: Jun. 26, 2014

(54) SOLID SUPPORT FOR ENDOTHELIAL CELL GROWTH

(71) Applicants: George Burke, Belfast (GB); Brian Meenan, Upper Lisburn (GB); Alan Brown, Belfast (GB)

(72) Inventors: George Burke, Belfast (GB); Brian Meenan, Upper Lisburn (GB); Alan Brown, Belfast (GB)

(73) Assignee: UNIVERSITY OF ULSTER, County Londonderry, Northern (IE)

(21) Appl. No.: 14/234,325

(22) PCT Filed: Jul. 30, 2012
(86) PCT No.: PCT/GB2012/051845

§ 371 Date: Mar. 10, 2014

(30) Foreign Application Priority Data
Jul. 29, 2011 (GB) 1113257.8

(51) Int. Cl. G01N 33/50; B29C 59/02; C12N 5/071

(52) U.S. Cl. CPC G01N 33/5044; C12N 5/069; B29C 59/022
USPC 435/29; 435/395; 435/288.4; 435/375; 264/293

(57) ABSTRACT

The invention relates to a solid support suitable for supporting endothelial cell growth which has one or more regions of microstructure incorporated onto the growing surface thereof as well as to such supports having endothelial cells attached thereto. The invention further relates to methods of culturing endothelial cells and directing tubule formation using these supports.

The present invention relates to solid supports which can be used to support cell growth, in particular which support angiogenesis. In particular, the present invention relates to solid supports which can be used to direct cell growth, in particular which promotes directional angiogenesis.

One of the major challenges in the field of Tissue Engineering is production of constructs which can integrate with the vascular system of the host in order to promote regeneration of the damaged tissue. Currently, the ability to produce a construct large enough to satisfy the clinical requirements relating to critical size defects is severely limited, due to the fact that the centre of the construct is unable to be supported by the host without vascularisation.

One way to mitigate this problem is to form a vascular network in the construct prior to implantation, and much research is being directed towards this goal. Existing approaches generally rely on biochemical stimulation of endothelial cells but this does not provide any control of the direction or orientation of cell growth or of angiogenesis.

Angiogenesis, the process of endothelial cell sprouting and growth of capillaries from pre-existing blood vessels, has been extensively studied experimentally and the molecular insights from these studies have lead to the development of therapies for wound repair, cancer, macular degeneration and ischemia. During this process, a series of cellular events occur. However, the complete angiogenic sequence has yet to be fully elucidated by either experimentation or modelling and hence numerous unknowns remain. However, what is known is that an endothelial cell from an existing vessel can become activated and this activated cell then starts to migrate into the surrounding extracellular matrix by degrading it; this unique, spindle-shaped cell is called the tip cell. Cells adjacent to the tip cell begin to proliferate and follow the tip cell, they are referred to as stalk cells. These processes result in formation of a sprout. This sprout is in the form of a capillary and moves towards a stimulus, in response to chemical cues, mechanical factors, and via a degree of random motility. Finally, the sprout joins an adjacent capillary and together these events define the process of sprouting angiogenesis.

Experiments have shown that differential VEGFR2 and PDGFβ gene expression, changes in proliferation, activation by threshold increases in VEGF protein levels and the cellular secretion of matrix degradation proteases like matrix metalloproteinases (MMPs) between individual endothelial cell types can all influence this process of sprouting angiogenesis, otherwise referred to as tubule formation.

While it is clear that under the right conditions angiogenesis will occur and capillaries will form, there is no methodology established to determine the direction of the sprouting process. Within a tissue engineered construct such directional control of sprouting angiogenesis (tubule formation) could enable better vascularisation and thus improve the chances of developing a clinically viable construct.

There is a need to develop a system where the spatial control of blood vessel formation allows tissue engineered constructs to be developed that would encourage vascularisation in a way that could be designed into scaffold architecture.

As well as in tissue engineering, a system which enabled some control over the direction of tubule formation would be of clinical value. In addition, such systems would have value as research platforms, e.g. models and assay methods. The control of angiogenesis, i.e. the growth of new blood vessels from pre-existing vessels, is a key topic of research in a number of fields e.g. vascular dysfunction and tumorigenesis. Angiogenesis inhibitors are used in the treatment of cancer and eye disease and angiogenic growth factors are being tested for their therapeutic use in a number of conditions, including in fields such as tissue engineering. In western countries, it is estimated that more than 184 million patients could benefit from anti-angiogenic therapy while more than 315 million could benefit from pro-angiogenesis therapy. More than $4 billion has already been invested in the development of angiogenesis based medicines, and it remains a focus of pharmaceutical development.

General research in the area to date has focused primarily on identifying and characterising angiogenic growth factors and inhibitors, but this approach has not considered the direct control of the direction or location of newly formed blood vessels created during the process.

Cell culture dishes or other solid supports that encourage directional angiogenesis would assist researchers in their understanding of the processes of angiogenesis. Such supports could provide a way to better control and regularise angiogenesis and thereby aid comparisons between different agents and experiments. Production of diagnostic cell culture substrates, or arrays, where tubules would form in specific areas and in specific directions would have value in the development of automated, high-throughput assays and screening methods for the development of angiogenic and anti-angiogenic drugs.

The present inventors have developed a substrate which functions as a solid support for endothelial cell growth and, through the provision of a non-uniform surface, enables the directional formation of tubules.

Thus, in a first aspect, the present invention provides a solid support suitable for supporting endothelial cell growth which has one or more regions of microstructure incorporated onto the growing surface thereof. Typically the regions of microstructure provide enhanced adherence for endothelial cells. Preferably, the adherence for endothelial cells is enhanced as compared to the regions without microstructure. Typically, where cells reach confluence on the microstructure the resultant cell density is greater than or about 8×10⁴ cells per cm². On the unfeatured areas, the cell density is preferably not greater than 1×10⁴ cells per cm², even more preferably the cell density is not greater than 1×10³ cells per cm².

The solid supports of the invention have a hydrophobicity which allows for directed tubule growth. Hydrophobicity is conveniently measured, as described in the Examples, in a wettability test which provides a static water contact angle (SCA) in degrees, e.g. using a CAM200 system. If the SCA is too high then there is poor cell adhesion but if it is too low then there is no discrimination between the regions as adherence is possible over the whole surface. Preferably the SCA of the support is greater than 80°, more preferably 80-88°.

The microstructure incorporates parts or areas or features which are raised, or possibly depressed, relative to the remainder of the growing surface of the solid support. Thus, the microstructure(s) provide a non-uniform growing surface. These structures are ‘micro’ structures and in particular will typically have a height (at the highest point) or depth (at the lowest point) which is 50 nm to 20 μm from the base level of the growing surface of the solid support, more usually 100 nm to 20 μm, preferably 0.25-10 μm. The regions without microstructure(s) have surface chemistry and topography such that cells do not readily adhere in these regions. This surface chemistry and topography is such that it creates a surface that is hydrophobic enough to reduce cell adhesion. This control of wettability can be as a result of defined surface roughness, surface chemistry or a combination of both. In the case of hot-embossed tissue culture treated polystyrene, a combination of a reduction of the surface roughness and a reduction in the surface oxygen concentration, combine to have this effect. Hot embossing processes as described in the Examples can conveniently provide this effect.

The “growing surface” of the solid support is a surface which is adapted to support cell growth after cells have been introduced thereto, e.g. by the standard process or step of cell seeding. This surface will typically include regions which are not intended to support significant cell growth according to the techniques of the present invention, i.e. regions other than the regions of microstructure to which the endothelial cells preferentially adhere.

The ‘regions of microstructure’ may be ridges, grooves, pillars or other arrangements. The pattern of these microstructures typically provides a stripe (or column) on the support surface and it has been shown that endothelial cells preferentially grow and align along this stripe and that angiogenesis takes place, generating a tubule. The microstructures are preferably arranged so that each individual ridge or groove is roughly perpendicular to the longitudinal axis of the stripe and that the ridge or grooves are arranged in parallel. Thus the stripe preferably has a width equal to the length of each ridge or groove. As such, angiogenesis preferably takes place perpendicular to the individual ridges or grooves. Any individual solid support may have a single stripe or several stripes, e.g. 2-100. Stripes may be formed from microstructures other than ridges or grooves, e.g. from raised pillars. Thus, the regions of microstructure are not flat but have a topography which preferably provides edges and a plurality of horizontal and vertical faces.

Where the region of microstructure is a pillar, the pillars would preferably be 1 to 10 μm in diameter. More preferably, the pillars would be 5 μm in diameter. The pillar would also preferably be produced in a hexagonal array, spaced about 5 μm apart from one another. These pillar arrangements could, as with the ridges described above, be arranged into stripes that are 50 μm to 400 μm wide, preferably 100 μm to 300 μm wide.

In a further embodiment of the invention, the stripes as described above would be arranged in an interlinking formation. Such a formation could be used to encourage tubule branching and thus each stripe or stripe part may not be parallel to the other stripes.

Where a solid support has more than one such region, these regions will preferably be arranged in parallel to one another. More importantly, they are preferably arranged such that endothelial cells growing in one region will not physically contact the cells in other regions. Typically therefore each region will be spaced at least 50 μm, preferably at least 100 or 150 μm, e.g. around 200 μm apart from each other.

The regions e.g. stripes themselves will preferably have an average width of between 100 and 400 μm, preferred average widths are between 150 and 250 μm, e.g. around 200 μm.

In other embodiments less strict geometric arrangements of the regions, stripes and so on may be provided, to allow for patterns of tubule growth which more closely follow natural tubule development. Thus connecting tubules and bifurcations can be enabled through appropriate patterning of the regions of microstructure. Networks may therefore be provided but still with spacing between the regions of microstructure so a network can be designed.

Where a region is made up of ridges or grooves, or effectively ridges and grooves, each ridge or groove will typically have a width between 1.5 and 60 μm preferably between 2 and 40 μm. However widths as small as 100 nm may be used. The widths need not be the same throughout the region but typically will be. Likewise, the spacing between the ridges/grooves need not be uniform but typically will be. The spacing distance for each ridge or groove will be of a similar size to the above-described width of each ridge/groove. Preferably ridges or grooves at the narrower end of the above width range will be closer together and wider ridges or grooves will be spaced further apart, most preferably the width of each ridge or groove will be approximately equal to the spacing between them. Ridges will conveniently be 0.25-10 μm high, preferably 0.4-5 μm in height.

The solid support itself may be made of any of the plasticware or glassware typically used for cell culturing or any other material on which the specific microstructed environment can be formed. Preferably, the solid support will maintain the required surface chemistry and topography in the non-structured regions that results in poor cell adhesion, such as by reduced surface roughness and reduced polar functional groups at the substrate surface. The support will preferably be made of a polymeric (including co-polymeric) material. Plastic supports are particularly preferred. Preferred materials include polystyrene, optionally tissue culture treated polystyrene (TCPS). The supports may be shaped as a dish, plate, array, flask, chip etc., depending on the use to which it is being put. Suitable supports may be made of poly lactic acid/glycolic acid (PLGA) and other polymers such as polylactic acid (PLA) and polycaprolactone (PCL) or co-polymers thereof. Suitable supports include Costar® culture plates of Corning and TOPS Sarstedt T-175 flasks. Other suitable materials include ceramics such as hydroxyapatite or bioglass or metal.

The support may also be irregularly shaped, and may not be flat, i.e. it may provide a 3D surface, particularly when it is designed to provide a scaffold for tissue engineering.

In a further aspect, the present invention provides a solid support as defined herein having endothelial cells attached thereto, preferably the majority of the endothelial cells (e.g. more than 55, 60, 70, 75 or 80%) on the solid support are attached to the regions of microstructure. In preferred embodiments, the endothelial cells have formed a tubule.

Various techniques exist for the formation of regions of microstructure on the growing surface of the solid support. The surface may be etched, embossed or coated; the solid support may alternatively be moulded from the outset to provide the required microstructure. Preferably the microstructures are generated by hot embossing of a plastic solid support, e.g. using a silicon or nickel stamp fabricated by photolithography and etching processes. The plastic substrate and master (stamp) are heated to a temperature that is just above the glass transition temperature of the substrate. At this point the master and substrate are pressed together with a controlled, uniform pressure. The master and substrate are then cooled to below the glass transition temperature before being separated. The result is that the features on the master are replicated in negative on the polymer substrate. This process is most often performed in a closed chamber, where the environment can be controlled, often including the use of inert gases and/or a vacuum. The embossing temperature is preferably just above the glass transition temperature of the polymer substrate, e.g. 105-110° C. for polystyrene. Thus, in a further aspect, the present invention provides a method of producing a solid support as defined herein, said method comprising:
(i) heating a plastic support substrate and stamp to a temperature that is just above the glass transition temperature of the substrate;
(ii) applying the stamp to the substrate under pressure;
(iii) cooling the stamp and the substrate below the glass transition temperature; and
(iv) separating the stamp from the support substrate to release the solid support.

The supports described herein have various utilities. They may be used as scaffolds for tissue engineering where it is desired to generate a functioning vasculature for use in vivo. Alternatively, the supports may be used as a culturing dish or surface, for example in assays relating to angiogenesis. Thus, in a further aspect, the present invention provides a method of culturing endothelial cells which comprises applying endothelial cells to a solid support as defined herein and then culturing the cells on said solid support. The cells may be cultured to confluence on the microstructure regions and then for several days (e.g. 2-14 days), as required for tubule formation, thereafter. Total incubation times may be 7-21 days. Suitable conditions for cell culturing are well known in the art and include control of temperature, nutrients, pH etc., e.g. at 37° C. with 5% CO₂, preferably at pH 7.4. The initial application of cells, the seeding step, may allow for 100,000 to 2,000,000 cells/cm² of support surface, typically 500,000 to 1,4000,000. In another preferred embodiment, the seeding step may allow for 10,000 to 200,000 cells/cm² of support surface, typically 50,000 to 150,000. The whole support surface may be seeded.

Any endothelial cells may be used, e.g. aortic endothelial cells or cardiac microvascular endothelial cells. The cells are preferably human but may be from other mammals or other animals e.g. they may be bovine or murine e.g. Bovine Aortic Endothelial Cells or Dermal Microvascular Endothelial Cells.

Various methods for inspecting or monitoring cell growth exist, preferred techniques involve Scanning Electron Microscopy, Confocal Laser Scanning Microscopy, live-cell video microscopy, Transmission Electron Microscopy and gene expression analysis, e.g. of the vascular endothelial growth factor (VEGF) receptor, (e.g. using Real Time Q-PCR). It may be helpful to use labelled antibodies, e.g. anti-VEGF receptor antibodies to assist in visualisation of angiogenesis.

In a preferred embodiment the present invention provides a method of promoting vascularisation on a three dimensional scaffold wherein said scaffold comprises a solid support as defined herein and endothelial cells are applied to said construct and cultured thereon.

The inventors have shown that, after tubule formation, they are able to detach the tubule from the solid support. Thus, in a further embodiment, the invention provides a method of culturing endothelial cells described above, wherein, after the tubule has formed, it is detached from said solid support. The tubule construct is preferably suitable for implantation in the body and may be a medical device or a tissue construct (e.g. bone). As shown in the Examples, the methods of the present invention preferably enable the generation of a tubule with a lumen.

In a further aspect, the invention provides an endothelial tubule or a vascular network which has been grown on a solid support as defined herein and/or in accordance with a method as defined herein.

The supports and methods of the invention can also be used in screening and assaying contexts. The supports and methods are suited to automation and high throughput screening techniques. In a further aspect the present invention provides a method of identifying or evaluating an agent which can modify angiogenesis, said method comprising applying a test agent and endothelial cells to a solid support as defined herein, culturing the cells on the solid support and monitoring tubule formation on said solid support. The performance of the test agent can be compared to negative or positive controls or to other candidate agents to evaluate the effect the test agent has on tubule formation, i.e. on angiogenesis. The test agent may be agonist or antagonist of angiogenesis or influence angiogenesis/tubule formation in other ways.

The methods and supports of the invention provide a platform for evaluation. In such methods, the solid support is conveniently arranged as an array, or a series of arrays, e.g. chips or multi-well plates, this allows high throughput screening of a library or other selection of agents and automation. The solid support may be of dimensions such as are typically used as microscope slides and may be analysed using a fluorescent microscope and motorised stage. Software exists or can be designed which allows for image analysis so the agents could be evaluated in an automated way. Visual inspection of tubule formation may be preferred in other embodiments.

Alternatively viewed, the present invention provides a method of directing angiogenesis, which method comprises applying endothelial cells to a solid support as defined herein and then culturing the cells on said solid support. In a still further aspect the present invention provides a method of directing tubule formation within a population of endothelial cells, said method comprising applying a population of endothelial cells to a solid support as defined herein and culturing the cells on said solid support. Suitable culturing conditions are described above and in the Example. As described herein, tubule formation and angiogenesis may be “directed” through the patterning of the regions of microstructure on the surface of the solid support. This patterning allows for control over the direction of tubule growth.

The invention also provides, in a further aspect, the use of a solid support as defined herein for directing angiogenesis or for directing tubule formation within a population of endothelial cells.

The invention will now be further described in the following non-limiting Example in which:

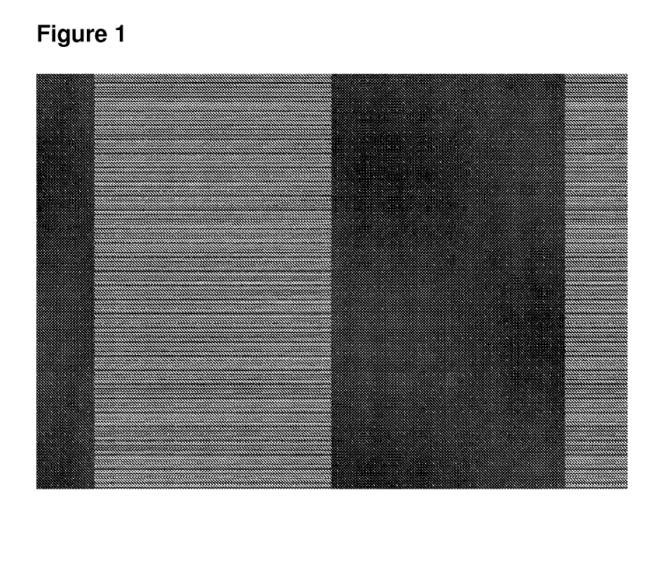

FIG. 1 is a CAD drawing photograph of a polystyrene solid support of the invention showing a stripe of ridges which are 200 μm long, 3.2 μm wide and 0.5 μm high. The spaces between ridges in the column are also 3.2 μm.

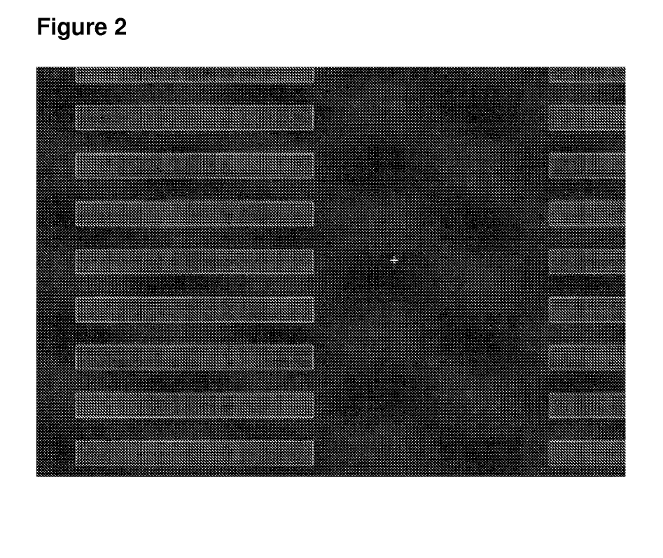

FIG. 2 is a CAD drawing photograph of a polystyrene solid support of the invention showing a stripe of ridges which are 200 μm long, 32 μm wide and 3 μm high. The spaces between ridges are also 32 μm.

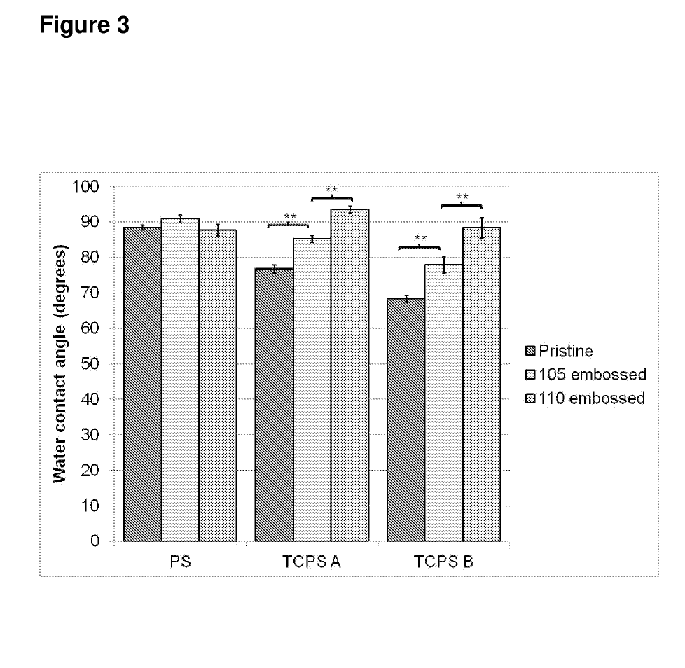

FIG. 3 is a graph showing the static contact angle measurements of pristine polystyrene (PS), and two different samples of TOPS (A & B). Measurements were carried out on samples that were either unembossed, embossed at 105° C., or embossed at 110° C. The higher the value is, the more hydrophobic the surface is (i.e. the more pronounced the curvature of the water droplet is). Error bars represent the standard error of the mean., ** denotes significance at p<0.01.

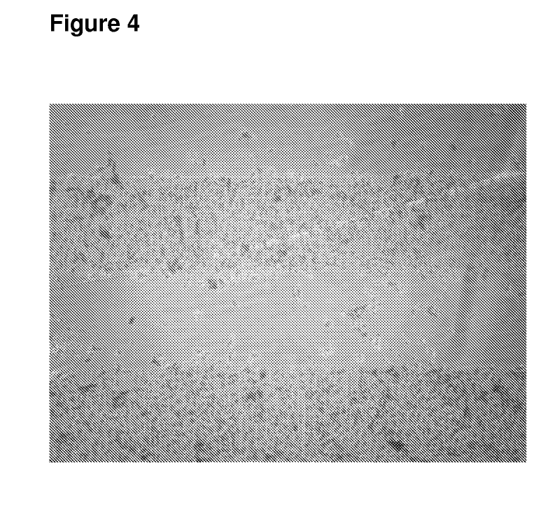

FIG. 4 is a phase contrast optical micrograph showing endothelial cells adhering to the micro-structured regions of the polystyrene solid support structure and showing little or no adherence to the unstructured regions.

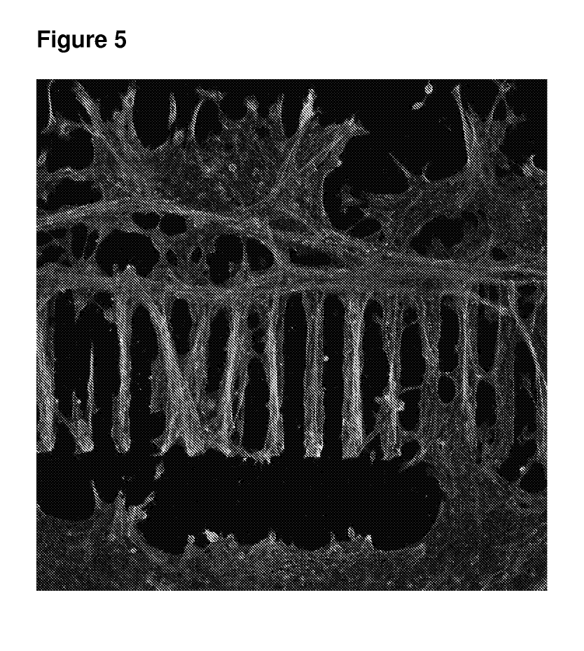

FIG. 5 is a confocal laser scanning micrograph showing endothelial tubules formed on 13 parallel vertical stripes of 200 μm long, 3.2 μm wide horizontal ridges.

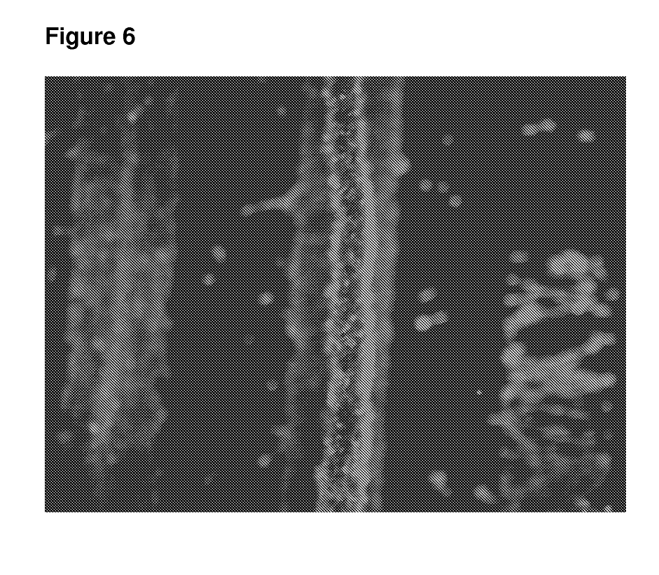

FIG. 6 is a fluorescent micrograph of endothelial tubules formed on a stripe of 200 μm long, 3.2 μm wide ridges.

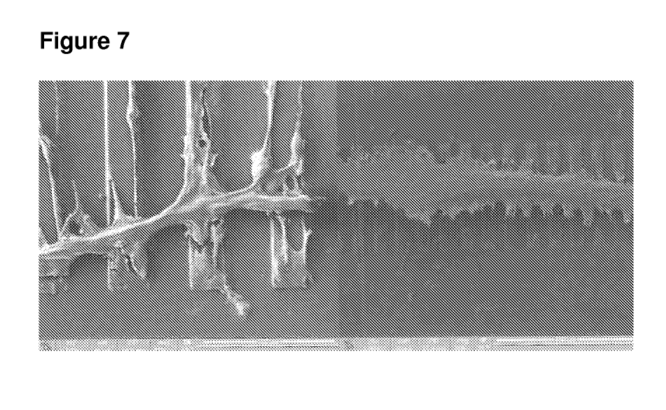

FIG. 7 are scanning electron micrographs of endothelial tubules formed on a stripe of 200 μm long, 32 μm wide ridges.

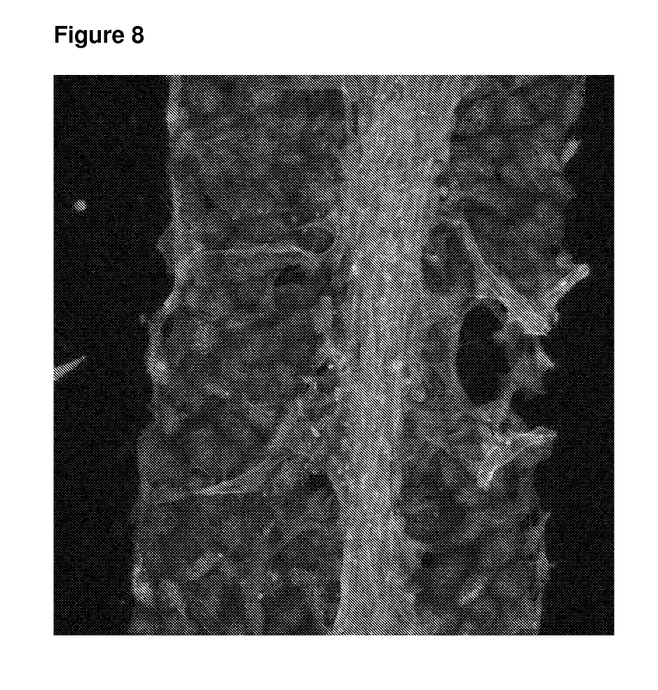

FIG. 8 is a confocal micrograph of tubule formed by BAECs on a 200 μm stripe of 3.2 μm wide ridges.

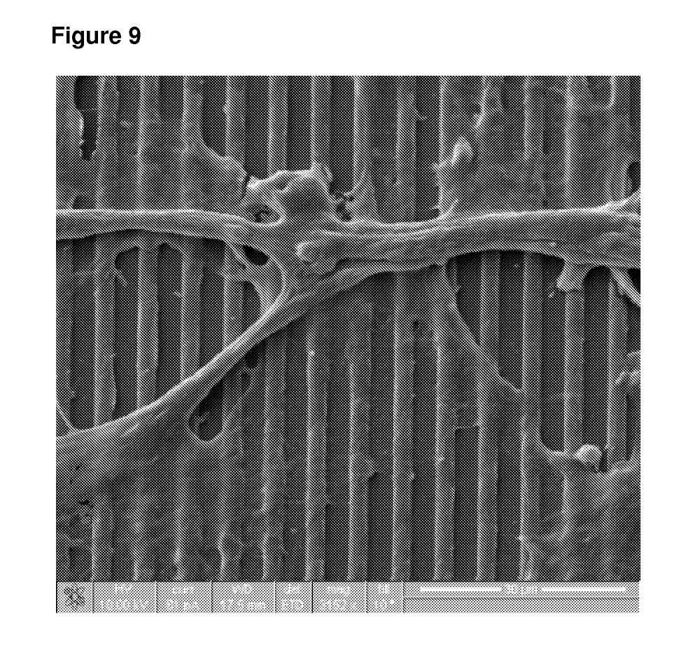

FIG. 9 is a scanning Electron micrograph of a tubule formed by BAECs on a 200 μm stripe of 3.2 μm wide ridges. The bifurcated tubule is formed perpendicular to the direction of the ridges on a single ridge array.

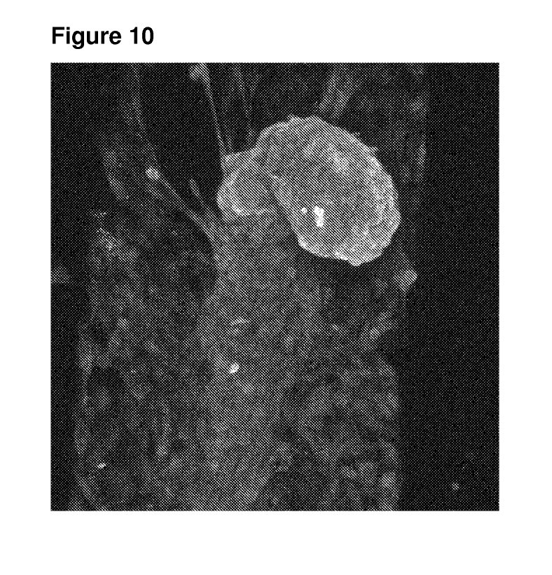

FIG. 10 is a two dimensional projection of CLSM image stack showing a single tubule detaching at one end from the microstructured array.

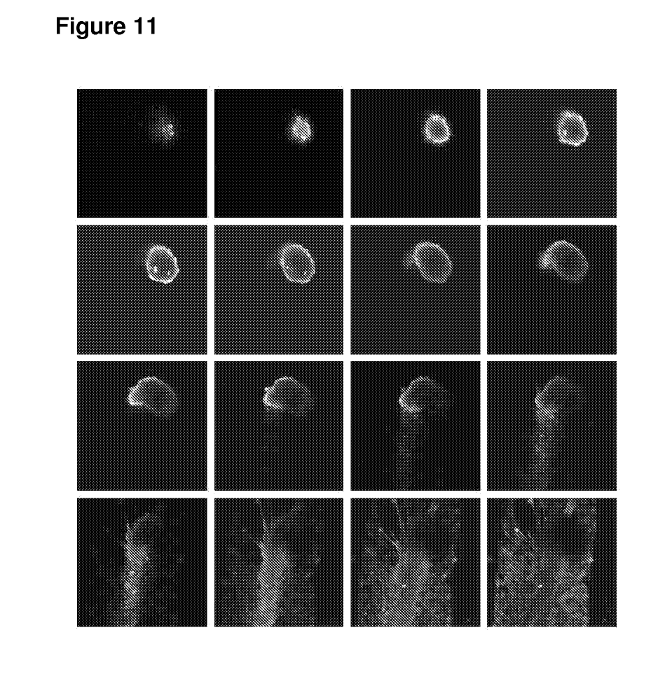

FIG. 11 is a Montage of CLSM image slices showing a single tubule detaching at one end from the microstructured array and showing the presence of the lumen.

### EXAMPLE 1

Stripes of 200 μm long, 3.2 μm wide and 0.5 μm high ridges with a space between the ridges of 3.2 μm, and stripes of 200 μm long, 32 μm wide and 3 μm high ridges with a space between the ridges of 32 μm were formed in tissue culture treated polystyrene flasks by hot-embossing, using a silicon stamp fabricated by photolithography and etching processes (see FIGS. 1 and 2). The solid supports which were embossed were standard TOPS flasks (Sarstedt T-175).

Primary Bovine Aortic Endothelial Cells (BAECs) were seeded at cell densities between 50,000 and 90,000 cells per cm² of the surface and were cultured on these surfaces for several days after they had reached confluence. Cells were cultured in Minimum Essential Media (MEM), supplemented with 10% Foetal Bovine Serum and including antibiotic/antimicotic. Incubation at 37° C. and 5% CO₂ was for 2 weeks in total and the culturing media was changed every 3-4 days.

After culturing, samples for scanning electron microscopy were fixed in 2.5% Gluteraldehyde for 30 minutes. After rinsing, secondary fixation for 10 minutes in 1% Osmium Tetroxide was performed. Samples were again rinsed and then dehydrated using a series of graded alcohols. 25% ethanol, 50% ethanol, 75% ethanol, 90% ethanol and 100% ethanol (twice) were added for 10 minutes each. Secondary dehydration in 50% HMDS in ethanol and then 100% HMDS for 10 minutes was performed. Samples were allowed to air-dry overnight before being coated in 20 nm of gold in a polaron sputter coater.

Samples for confocal microscopy were labelled as follows. Fixation was performed in 4% Paraformaldehyde, containing 0.1% Triton-X-100 for 20 minutes. After rinsing, samples were incubated with a primary antibody (either Anti-βTubulin, Anti-Flt-1 or Anti-Flk-1) for 2 hours at room temperature. After rinsing, samples were incubated with a fluorescent secondary antibody, conjugated with AlexaFluor 546. In most cases, samples were then incubated with AlexaFluor 488 conjugated Phalloidin to label the actin cytoskeleton and DAPl to label cell nuclei. Once labelled, samples were mounted using Vectashield mounting medium, cover-slipped and sealed with nail varnish.

The cells were found to have formed tubules running perpendicular to the patterned ridges (see FIGS. 4 to 9). The hot embossing was carried out using an automated hot embossing system (EVG 520HE, EV Group, Scharding, Austria) at a temperature of 105° C. and a pressure of 20 kN. All embossing processes were performed under a vacuum of <1 mbar.

### EXAMPLE 2

### The Effect of Embossing Conditions on Directional Tubule Formation

### Introduction

In previous investigations of directional tubule formation, one of the key initial observations is that cells adhere preferentially to arrays of micro-scale features, when compared with flat areas, where features are not present. Moreover, when this preferential cell attachment is not seen, then directional tubule formation is also not readily observed. This study was performed to investigate the effect of embossing conditions on directional tubule formation effects.

### Methods

Circular TCPS samples that were 100 mm in diameter were cut from Sarstedt T-175 tissue culture flasks using a hot wire cutter. Stripes of 200 μm long, 3.2 μm wide and 0.5 μm high ridges with a space between the ridges of 3.2 μm, and Stripes of 200 μm long, 32 μm wide and 3 μm high ridges with a space between the ridges of 32 μm were formed in Tissue culture treated polystyrene flasks by hot-embossing, using a silicon stamp fabricated by photolithography and etching processes. The hot embossing was carried out using an automated hot embossing system (EVG 520HE, EV Group, Scharding, Austria) at a temperature of either 105° C. and a pressure of 20 kN, or a temperature of 110° C. and a pressure of 10 kN. All embossing processes were performed under a vacuum of <1 mbar.

The wettability of the embossed samples was then measured using a CAM200 static contact angle measuring system. At least four spots were measured on each of at least three samples per experimental split. The water droplets are formed in the flat regions between the micro-scale (microstructure) stripes formed in the embossing process.

Cell attachment and preferential adhesion were evaluated by sterilising the substrates in 70% ethanol and allowing them to air-dry. Primary Bovine Aortic Endothelial Cells (BAECs) were seeded as described in Example 1.

After culturing, the samples were imaged using optical microscopy, scanning election microscopy and confocal microscopy, as described in Example 1.

### Results

Embossed and unembossed samples were tested. FIG. 3 shows the contact angles of pristine polystyrene, and two different samples of TOPS, either unembossed or embossed at 105° C. or embossed at 110° C. Sample A is an old batch of TCPS which was previously shown to give good results in these guided tubule formation experiments. TCPS B is a new batch of the same material which has not shown the same degree of success in these experiments. The contact angle of TCPS A after embossing is significantly higher than TCPS B. It is therefore suggested that cells will not generally adhere well to the more hydrophobic embossed TCPS A, but they will still adhere to the micro-patterned regions thereof. TCPS B, being more hydrophilic, does not have the same degree of selective adhesion, i.e. lacks the necessary distinction between hydrophobic and hydrophilic zones, and therefore is less successful in these experiments.

From these results, we predict that for the TCPS substrate to be of use in tubule formation, the embossing process must lead to a substantial increase in surface hydrophobicity, so that the cells will only grow on the micro-patterned surface.

Embossing at higher temperature (110° C.) significantly increased the hydrophobicity of the TCPS B substrate, suggesting that this substrate may also be of use for tubule formation.

BAECs were confluent on the TCPS surface after 24 hours. Fewer cells were adhered to many of the microstructured surfaces, primarily due to the fact that preferential adhesion was observed for both the 3.2 μm wide ridges and the 32 μm wide ridges, see FIG. 4. What is clear from FIG. 4 is that the cells adhere to the top surfaces of the features.

Scanning electron micrographs, shown in FIGS. 7 and 9 show the tubules forming on 3.2 μm wide ridge structures. Preferential attachment of cells to the microstructured regions can also be seen and while the tubules tend to form along the stripe of microstructured ridges, some interconnecting tubules can also be seen. The attachment of cells to the top surface of the microstructures is also evident.

### EXAMPLE 3

### Microscopical Investigation of the Nature of Directional Tubule Formation

### Methods

Cells were seeded and cultured as in Example 2 on a variety of different embossed and unembossed surfaces. After 15 days, samples were fixed in 4 paraformaldehyde containing 0.1% Triton X-100 for 20 minutes. After washing, samples were incubated in a primary antibody directed against either VEGF receptor 1 (Flk-1) or VEGF receptor 2 (Flt-1) for 1 hour at 37° C. A fluorescent secondary antibody conjugated with AlexaFluor 546 was then applied for 45 minutes at 37° C. Samples were also stained with AlexaFluor 488 conjugated phalloidin and DAPI, before being mounted using Vectashield, cover slipped and sealed with clear varnish.

Image stacks were obtained by CLSM. These image stacks were rendered as two-dimensional images using Image examiner (Zeiss) and also processed using Volocity (Perkin Elmer) image analysis software in order to view the three-dimensional structure formed.

### Results

The images presented in FIGS. 8 and 10 show both preferential adhesion to the micro-patterned areas and directional tubule formation along the stripes of ridges. All these images were taken from material that had been embossed at 110° C. The detachment of tubules from the substrate was seen in a number of areas. One of these areas was then analysed using Volocity software (FIG. 10). Images from this can be seen in the montage of images in FIG. 11.

These images demonstrate that the structures that are formed by endothelial cells are indeed tubular and form a recognisable lumen. The diameter of the lumen is 30-40 μm.

By using both CLSM and advanced image analysis software it has been shown that microstructured, hot embossed surfaces can produce directional tubule formation, and that the tubules formed can be seen to have a large lumen.

1. A solid support suitable for supporting endothelial cell growth which has one or more regions of microstructure incorporated onto the growing surface thereof.

2. The solid support of claim 1, wherein said regions of microstructure provide enhanced adherence for endothelial cells when compared to the regions without microstructure.

3. The solid support of claim 1, wherein said regions of microstructure are in the form of stripes on said growing surface.

4. The solid support of claim 3 wherein said stripes have an average width of between 100 and 400 μm.

5. The solid support of claim 3 wherein said stripe comprises (a) multiple ridges or grooves that lie perpendicular to the longitudinal axis of said stripe or (b) pillars.

6. The solid support of claim 5 wherein the (a) ridges or grooves are 1.5-60 μm wide or (b) the pillars are 1 to 10 μm in diameter.

7. The solid support of claim 1, wherein said regions of microstructure have a height, at the highest point, or depth, at the lowest point, which is 50 nm to 20 μm from the base level of the growing surface of said solid support.

8. The solid support of claim 7, wherein said regions of microstructure have a height, at the highest point, or depth, at the lowest point, which is 100 nm to 20 μm from the base level of the growing surface of said solid support, preferably 0.25 to 10 μm from the base level of the growing surface of said solid support.

9. The solid support of claim 1 which generates a static water contact angle of greater than 80°, preferably 80-88°.

10. The solid support of claim 1, which is made of a material selected from polystyrene, polylactic acid, polycaprolactone, co-polymers of said polymers, ceramic or glass.

11. The solid support of claim 1 having endothelial cells attached thereto.

12. The solid support of claim 11 wherein the majority of the endothelial cells on the solid support are attached to the regions of microstructure.

13. The solid support of claim 11 wherein the endothelial cells have formed a tubule.

14. A method of producing a solid support as claimed in claim 1, said method comprising:
(i) heating a plastic support substrate and stamp to a temperature that is just above the glass transition temperature of the substrate;
(ii) applying the stamp to the substrate under pressure;
(iii) cooling the stamp and the substrate below the glass transition temperature; and
(iv) separating the stamp from the support substrate to release the solid support.

15. A method of culturing endothelial cells comprising;
(i) applying endothelial cells to a solid support as defined in claim 1; and
(ii) culturing said endothelial cells on said support.

16. The method of claim 15 further comprising the following steps:
(iii) culturing said endothelial cells to confluence on the regions of microstructure; and
(iv) further culturing the cells until tubule formation.

17. (canceled)

18. A method of identifying or evaluating an agent which can modify angiogenesis, said method comprising:
(i) applying said agent and endothelial cells to a solid support as defined in claim 1;
(ii) culturing the cells on the solid support; and
(iii) monitoring tubule formation on said solid support.

19. An array, chip or multi-well plate comprising multiple solid supports as defined in claim 1 suitable for use in high throughput screening.

20. A method of directing angiogenesis or tubule formation within a population of endothelial cells, said method comprising:
(i) applying said endothelial cells to a solid support as defined in claim 1; and
(ii) culturing the cells on said solid support.
